(19) United States
(12) Patent Application Publication
(10) Pub. No.: US 2025/0101352 A1
(43) Pub. Date: Mar. 27, 2025

(54) SYSTEMS AND METHODS FOR DECARBONIZATION THROUGH BIOMASS FEEDSTOCK, AN ENERGY GENERATING SYSTEM, AND A BIOPROCESSING FACILITY

(71) Applicant: POET Research, Inc., Sioux Falls, SD (US)

(72) Inventors: David Charles Carlson, Brandon, SD (US); Chris Dickman, Sioux Falls, SD (US)

(21) Appl. No.: 18/891,814

(22) Filed: Sep. 20, 2024

Related U.S. Application Data
(60) Provisional application No. 63/540,849, filed on Sep. 27, 2023.

(51) Int. Cl. C12M 1/107; B01D 53/02; B01D 53/14; B01D 53/62; C12M 1/00; F01K 11/02

(52) U.S. Cl. CPC C12M 21/04; B01D 53/62; C12M 23/36; C12M 23/40; C12M 43/08; B01D 53/02; B01D 53/1475; B01D 2252/204; B01D 2257/504; B01D 2258/0283; F01K 11/02

(57) ABSTRACT

Methods of capturing atmospheric carbon-dioxide gas by generating energy via combustion of a biomass feedstock and providing at least a portion of the energy to a bioprocessing facility. The combustion of the biomass feedstock produces a flue gas having carbon-dioxide gas that can be captured. Related facilities.

### CROSS-REFERENCE TO RELATED APPLICATION

This nonprovisional patent application claims the benefit of U.S. Provisional Patent Application Ser. No. 63/540,849, filed on Sep. 27, 2023, wherein said provisional patent application is incorporated herein by reference in its entirety.

### BACKGROUND

The present disclosure relates to reducing the carbon intensity of a bioprocessing facility. Bioprocessing of feedstocks to produce various bioproducts is an increasingly important source of products including such things as feed, food, fuel, pharmaceuticals, and other chemicals. There is a continuing need for reducing the carbon intensity of bioprocessing facilities.

### SUMMARY

The present disclosure includes embodiments of methods and facilities adapted to capture atmospheric carbon-dioxide gas.

In one illustrative example according to the disclosure, a method includes:
generating energy via combustion of a biomass feedstock, wherein the combustion of the biomass feedstock produces a flue gas including carbon-dioxide gas;
providing at least a portion of the energy to a bioprocessing facility; and
capturing at least a portion of the carbon-dioxide gas in the flue gas.

In another illustrative example according to the disclosure, a facility includes:
at least one energy generating system configured to generate energy via combustion of a biomass feedstock and produce a flue gas including carbon dioxide, wherein the facility is adapted to capture at least a portion of the carbon-dioxide gas in the flue gas; and
a bioprocessing facility co-located with the at least one energy generating system, wherein the bioprocessing facility includes:
   a fermentation system configured to ferment a fermentable composition and generate at least one target biochemical and carbon-dioxide gas; and
   one or more systems configured to use energy for one or more processes in the bioprocessing facility, wherein at least one of the one or more systems are configured to receive at least a portion of the energy from the at least one energy generating system.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1 is a schematic showing an illustrative embodiment of a facility and process for capturing atmospheric carbon-dioxide gas;

FIG. 2A is a process flow diagram of a base scenario of carbon emissions for a corn-ethanol bioprocessing facility;

FIG. 2B is a process flow diagram of an illustrative embodiment of a corn-ethanol bioprocessing facility adapted to capture atmospheric carbon-dioxide gas of carbon emissions as compared to FIG. 2A;

FIG. 2C is a schematic showing an illustrative embodiment of a facility and process according to FIG. 2B; and

FIG. 3 is a schematic showing another illustrative embodiment of a facility and process for capturing atmospheric carbon-dioxide gas.

### DETAILED DESCRIPTION

At least one factor in climate change includes increasing levels of carbon-containing greenhouse gases in the atmosphere. One such gas is carbon-dioxide (CO2) gas, which is produced, e.g., when fuels are burned for transportation, heat, and/or electrical generation. When fossil fuels are burned, fossil carbon that has been locked underground for millions of years is released and increases the carbon concentration in the atmosphere. When plants grow, they absorb carbon from the atmosphere reducing the carbon concentration in the atmosphere. When plant-based materials are burned, the amount of carbon dioxide released is equal to or less than the amount of carbon dioxide absorbed during their growth resulting in no net increase in carbon in the atmosphere. Converting from a fossil fuel to a such a biogenic fuel results in energy consumption with a lower or no increase in atmospheric CO2 enabling a carbon neutral energy source. In some embodiments, capturing and sequestering CO2 can lead to a reduction in atmospheric CO2, thereby enabling a carbon negative process. In both cases, using a biogenic fuel reduces the carbon intensity of the bioprocessing facility as compared to if fossil fuel is the only source of fuel for generating energy. As used herein, a “biogenic fuel” such as biomass feedstock means a fuel source for which its carbon content is derived from biogenic carbon as compared to anthropogenic carbon. Biogenic carbon is considered to be carbon that has recently (within the last thousand to several million years) been a part of the natural carbon cycle consisting of living organisms and atmospheric carbon dioxide. Anthropogenic carbon, by contrast, is considered to be carbon that for recent history (the last thousand to million years) has been stored outside of the natural carbon cycle (such as in fossil energy resources such as coal, petroleum, or natural gas) and has been moved into the natural carbon cycle due to human activities such as burning fossil energy resources.”

The present disclosure relates to systems, facilities, and methods for decarbonization through biomass feedstock. Decarbonization means reducing the amount of carbon dioxide released into the atmosphere compared to a baseline. Non-limiting examples of decarbonization processes include carbon-neutral processes and/or carbon-negative processes.

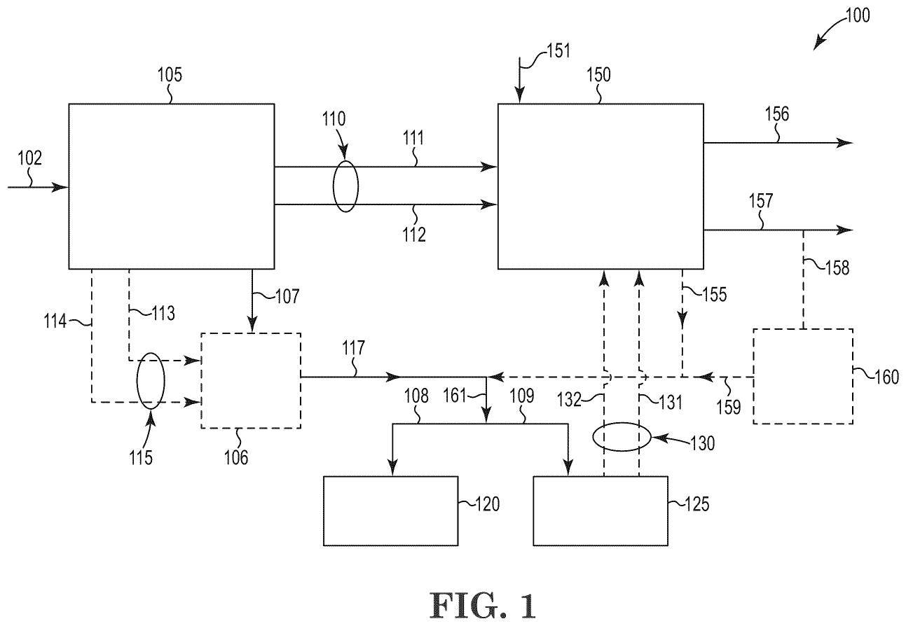

According to the present disclosure, systems and methods can be used for decarbonization by capturing atmospheric carbon-dioxide gas that is produced by generating energy via combustion of a biomass feedstock and/or that is produced by utilizing biomass feedstock in a bioprocessing facility. The combustion of the biomass feedstock produces a flue gas, and a bioprocess produces process gas, that includes carbon-dioxide gas. At least a portion of the energy can be provided to the bioprocessing facility and at least a portion of the carbon-dioxide gas in the flue gas and/or process gas can be captured, thereby reducing the carbon intensity of the bioprocessing facility as compared to if fossil fuel is the only source of fuel for generating energy. A non-limiting embodiment of a system according to the present disclosure will be described with respect to facility 100 in FIG. 1, where facility 100 is adapted to capture atmospheric carbon-dioxide gas.

Facility 100 includes at least one energy generating system 105 configured to generate energy 110 via combustion of a biomass feedstock 102 and produce a flue gas 107 that includes carbon dioxide gas. At least a portion of the energy produced by energy generating system 105 is provided to a bioprocessing facility 150.

Energy generating system 105 represents one or more energy generating systems that are co-located with and integrated with a bioprocessing facility 150 to provide energy 110 directly to the bioprocessing facility 150 instead of, or in addition to, energy from sources outside of facility 100 such as a grid (not shown). As shown in FIG. 1, energy generating system 105 can produce and deliver electrical energy 111 and/or thermal energy 112 at least to bioprocessing facility 150. In some embodiments, energy generating system 105 can produce and deliver energy to one or more other systems within facility 100 and/or export energy to one or more grids. For example, as discussed below, in some embodiments energy generating system 105 can produce and deliver energy 115 to the system 106, which is configured to separate at least a portion of the carbon-dioxide gas from the flue gas 107 to form stream 117 that is relatively more concentrated in carbon-dioxide gas as compared to flue gas 107.

Electrical energy 111 can be generated using steam and a power-generation system (“steam-generated power”). Steam-generated power can generate electrical power using a steam turbine system that receives steam from a steam-boiler system. Non-limiting examples of steam turbine systems include condensing turbine systems, non-condensing turbine systems, reheat turbine systems, extracting turbine systems, and combinations thereof.

A steam-boiler system can create steam at a temperature and pressure for a steam turbine system via combustion of biomass feedstock 102 and one or more oxidants, which produces thermal energy and a gaseous exhaust (flue gas 107).

In addition to, or instead of, producing electrical energy 111, energy generating system 105 can produce thermal energy 112 for use with one or more process streams and/or one or more systems in bioprocessing facility 150. Heat generated can be present in a liquid or gas medium and be used to heat one or more process streams in bioprocessing facility 150. Energy generating system 105 can generate heat by combustion of biomass feedstock 102 and one or more oxidants, which produces thermal energy 112 and flue gas 107.

In some embodiments, an energy generating system 105 can create heat via combustion so that the heat can be used to form steam, which can be used as thermal energy 112 to heat one or more process streams and/or generate electrical energy 111. A non-limiting example of such energy generating system 105 is known as a “combined heat and power facility,” which includes a steam boiler system that generates steam suitable for generating electrical energy 111 using a steam turbine system (discussed above). Non-limiting examples of an inlet pressure of steam suitable for generating electrical energy 111 in a steam turbine system include a pressure in a range from 200 pounds per square inch (psig) to 2000 psig, from 250 psig to 1000 psig, or even from 300 psig to 600 psig. Non-limiting examples of an inlet temperature of steam suitable for generating electrical energy 111 in a steam turbine system include a temperature in a range from 200° C. to 300° C., or even from 215° C. to 255° C. A combined heat and power facility can also deliver pressurized steam suitable for “process” steam that can be transported through a pipe system to one or more points of use in bioprocessing facility 150 (e.g., to an evaporator system and/or a distillation system and/or a dryer system). Non-limiting examples of steam pressure suitable for process steam include a pressure in a range from 50 psig to 200 psig, or even from 100 psig to 125 psig. Non-limiting examples of steam temperature suitable for process steam include a temperature in a range from 150° C. to 200° C., or even from 170° C. to 185° C. In some embodiments, steam exhausted from a steam turbine system (e.g., non-condensing turbine system) is controlled by a regulating valve so that the steam is at a temperature and pressure that permits the steam exhausted from the steam turbine system to be used as “process” steam for, e.g., an evaporator system and/or a distillation system.

As mentioned above, a biomass feedstock 102 can also be referred to as a “biogenic” fuel. Non-limiting examples of biomass feedstock 102 include renewable, solid fuel. Biomass feedstock 102 may come from the seed, sap, stems, and leaves of plants. A wide variety of plant-based feedstocks can be used according to the present disclosure such as agricultural residues (e.g., husks, stems, corn stover, sugarcane bagasse, straw), grasses, wood, and woody plants.

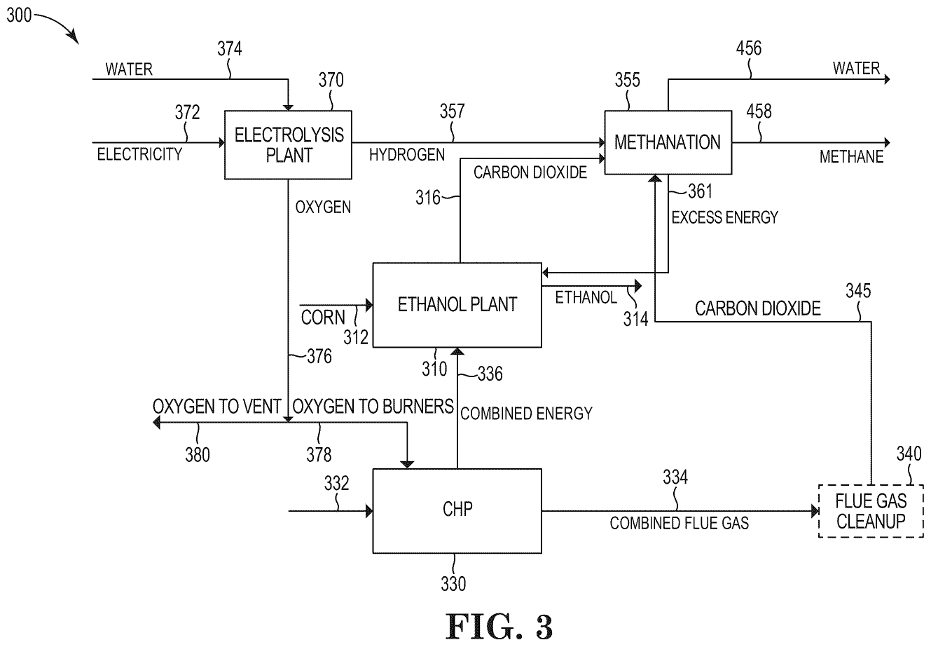

Oxidants have a relatively high oxidation potential. Non-limiting examples of gaseous oxidants include atmospheric oxygen, concentrated oxygen, and combinations thereof. Atmospheric oxygen is a component of atmospheric air, which includes about 78% nitrogen, 21% oxygen, and about 1% argon. Concentrated oxygen can be provided from a variety of sources having a variety of oxygen concentrations, an example of which is discussed below with respect to FIG. 3.

Combustion can produce a flue gas 107 having a composition that depends on the fuel, oxidant, and combustion conditions (e.g., temperature and amount of oxidant). For example, the ratio of fuel to oxidant can be selected to facilitate complete combustion. The reaction can primarily yield carbon-dioxide gas and water. Trace elements that may be present can also react to form common oxides such as sulfur dioxide, iron (III) oxide, and the like. If atmospheric air is used as a source for the oxidant oxygen, the nitrogen is generally not considered a combustible substance.

As mentioned, at least a portion of the energy 110 can be provided directly to a bioprocessing facility 150. Advantageously, the bioprocessing facility 150 can reduce or avoid obtaining energy from sources external to facility 100, such as from an external electrical grid, especially energy from sources that use fossil fuel, thereby reducing the relative carbon intensity.

As used herein, a “bioprocessing facility” refers to a facility that can produce one or more bioproducts by converting biomass feedstock via one or more physical processes, one or more chemical processes, one or more bioprocesses, and combinations thereof. Non-limiting examples of bioprocessing facilities include dry mills, wet mills, biofuel production facilities, pharmaceutical production facilities, soy processing facilities, breweries, bakeries, and the like.

A bioproduct refers to a product derived from a biological, renewable resource. For example, a bioproduct can be a component of biomass feedstock that is liberated from the biomass feedstock (e.g., corn oil from corn grain) and/or can include a chemical (“biochemical” or “target biochemical”) that is produced by a biocatalyst (e.g., microorganism and/or enzyme) such as, for example, alcohol produced by yeast fermenting sugar. Non-limiting examples of bioproducts produced in a bioprocessing facility include one or more of fuel, feed, food, pharmaceuticals, beverages and precursor chemicals. In some embodiments, a bioproduct includes, among others, one or more monomeric sugars, one or more enzymes, one or more oils, one or more alcohols (e.g., ethanol, butanol, and the like), fungal biomass, amino acids, and one or more organic acids (e.g., lactic acid), and combinations thereof.

In some embodiments, one or more bioprocesses are carried out in a bioprocessing facility utilizing living cells (one or more microorganisms) and/or their components (e.g., enzymes produced by a microorganism) to obtain a desired bioproduct. Non-limiting examples of bioprocesses include one or more of hydrolysis (e.g., enzymatic hydrolysis), aerobic fermentation, or anaerobic fermentation. In some embodiments, a bioprocess includes saccharification and fermentation of a plant-based feedstock into a biofuel via enzymatic hydrolysis and yeast-based fermentation of the hydrolysate (e.g., yeast-based fermentation in a grain-to-ethanol biofuel facility).

As shown in FIG. 1, facility 100 includes a bioprocessing facility 150 that can receive at least one biomass feedstock 151. Bioprocessing facility 150 includes at least one fermentation system (not shown) that is configured to ferment a fermentable composition (also referred to as a fermentable mash) and generate carbon-dioxide gas in stream 155, at least one target biochemical 156, and one or more bioproducts 157.

Non-limiting examples of material to provide a fermentable composition include one or more of microorganisms, enzymes, carbon sources (e.g., biomass feedstock 151), aqueous compositions (e.g., fresh water, backset, etc.), nutrient sources (e.g., biomass feedstock), etc. In some embodiments, a feedstock can function as a carbon source and/or a nutrient source, and can be used to form a fermentable composition. A biomass feedstock can include one or more components that are utilized by a microorganism to produce one or more bioproducts via a bioprocess. Non-limiting examples of biomass feedstock may include, e.g., monosaccharides such as glucose and fructose, disaccharides such as sucrose and lactose, and more complex polysaccharides such as starch, cellulose, hemicellulose, and pectin. These biomass feedstocks may come from the seed, sap, stems, and leaves of plants. A wide variety of plant-based biomass feedstocks can be used according to the present disclosure such as sugar beets, sugar cane, grains, legumes, crop residues (e.g., husks, stems, corn stover, sugarcane bagasse, wheat straw), grasses, and woody plants. In some embodiments, biomass feedstock can be derived from corn, sorghum, wheat, rice, barley, soybean, rapeseed, oats, millet, rye, corn stover, straw, bagasse and the like. In some embodiments, a biomass feedstock can include whole ground grain (e.g., corn flour) formed via, e.g., a dry-grind process. In some embodiments, a biomass feedstock can include one or more cellulosic polysaccharides (e.g., grain kernel fiber, crop waste, wood, municipal waste, etc.), and combinations thereof.

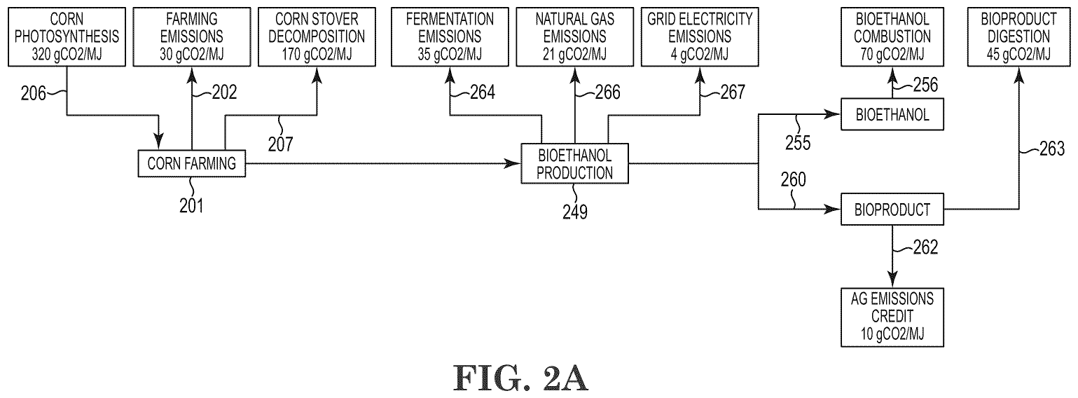

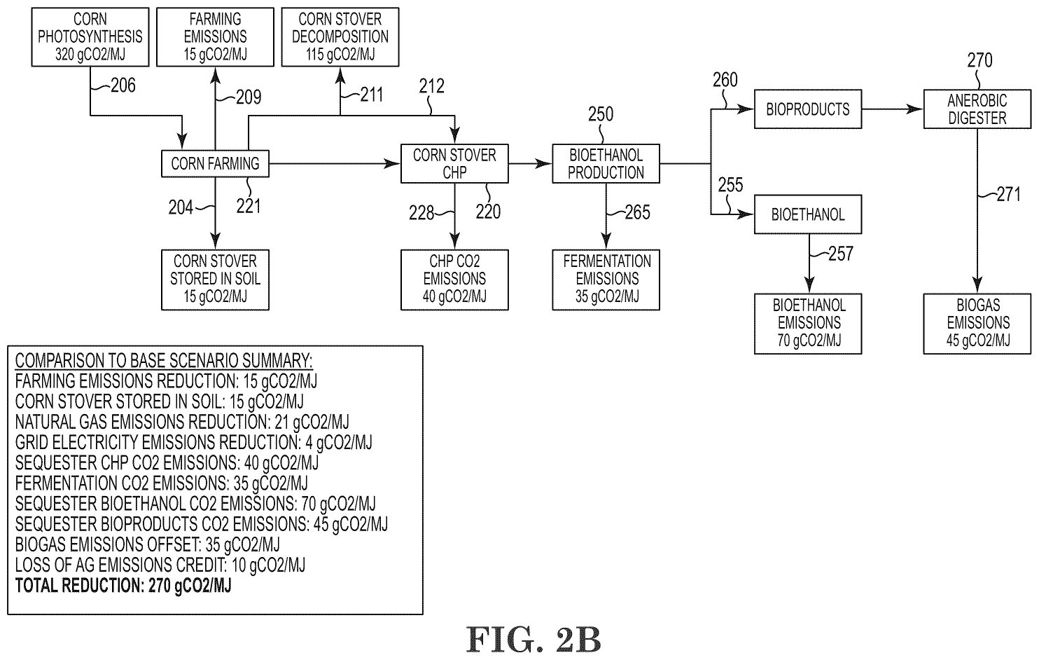

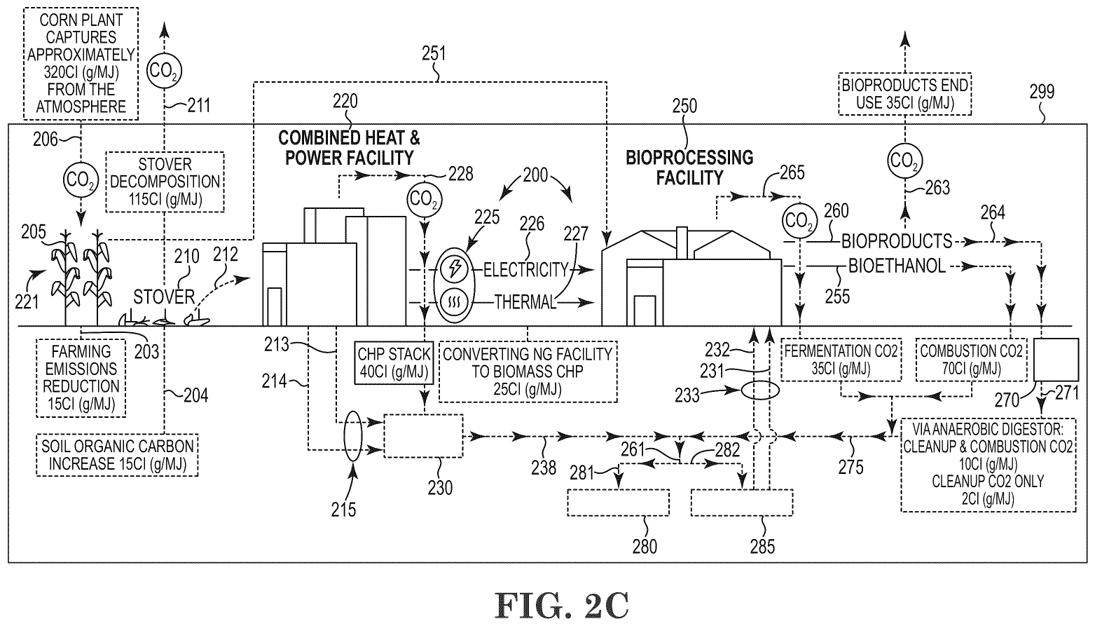

In some embodiments, a biomass feedstock 151 used in a fermentation system of bioprocessing facility 150 may be the same as biomass feedstock 102 used in energy generating system 105, or derived from the same biomass used for biomass feedstock 102. For example, as discussed below in connection with FIGS. 2A-2C, the biomass feedstock 212 used as fuel for energy generating system 220 includes corn stover left over from harvesting corn grain while bioprocessing facility 250 uses corn grain as biomass feedstock for producing target biochemical shown as ethanol (bioethanol) in stream 255.

In some embodiments, a fermentation system in bioprocessing facility 150 can include one or more feedstock systems to process biomass feedstock from one form into another form prior to fermentation. For example, a feedstock system can include one or more size-reduction devices to reduce the size of raw biomass feedstock such as grain and/or further reducing the size of ground grain that has previously been reduced in size. Methods for reducing the size of biomass feedstock, e.g., corn and/or previously ground corn, into fine particles prior to fermentation include dry methods such as passing corn through one or more hammer mills, ball mills and/or roller mills or wet methods such as passing a ground grain slurry through one or more mills such as a disc mill, roller mill, colloid mill, ball mill or other type of milling device.

In some embodiments, a ground biomass feedstock can be mixed with an appropriate amount of water (e.g., in a slurry tank) to form at least a portion of a fermentable composition (sometimes referred to as a mash). In some embodiments, whole ground corn can be mixed with liquid at about 20 to about 50 wt-% or about 25 to about 45 wt-% dry whole ground corn based on the total weight of the slurry. Whole ground corn can include starch, fiber, protein, oil, endogenous enzymes, amino acids, etc.

One or more exogenous microorganisms can be present in the fermentable composition of a fermentation system to produce beer that includes at least one or more biochemical bioproducts. Fermentation by a microorganism can produce biomass (e.g., single cell protein (SCP)), metabolites (e.g., alcohol such as ethanol; enzymes), and combinations thereof. Non-limiting examples of such microorganisms include one or more of ethanologens, butanologens, and the like. Exemplary microorganisms include one or more of fungi (e.g., yeast or filamentous), algae, or bacteria. For example, yeast may be used to convert the sugars to an alcohol such as ethanol. Suitable yeast includes any variety of commercially available yeast, such as commercial strains of Saccharomyces cerevisiae.

Optionally, one or more additional, exogenous materials may be utilized in a fermentable composition. Non-limiting examples of such materials include one or more of enzymes, phosphate, citric acid, ionic additives, and the like.

A fermentation system can include one or more vessels that are adapted to expose a fermentable composition to conditions suitable for converting sugars such as glucose to one or more bioproducts. As used herein, a “vessel” refers to any vessel that permits a bioproduct to be formed from a microorganism via fermentation. In some embodiments, a vessel can refer to a bioreactor adapted or configured to expose a fermentable composition to fermentation conditions. Non-limiting examples of vessels that can expose a fermentable composition to fermentation conditions include fermenters, beer wells, and the like. Two or more vessels may be arranged in any desired configuration such as parallel or series.

A fermentation system is configured to expose fermentable composition to fermentation conditions so that one or more microorganisms can convert one or more components in the fermentable composition such as sugars into a beer that includes one or more target bioproducts. Fermentation conditions include one or more conditions such as pH, time, temperature, aeration, stirring, and the like.

The pH of a fermentable composition can be at a pH that helps a microorganism produce one or more target bioproducts in a desired quantity. In some embodiments, the pH is greater than 3.5, e.g., from 3.5 to 7, from 3.5 to 5.5, or even from 3.5 to 4.5. Techniques for adjusting and maintaining pH include, e.g., adding one or more acidic materials and/or adding one or more basic materials.

With respect to temperature and time, the contents of a fermentable composition can be maintained at temperature for a time period that helps a microorganism produce one or more target bioproducts in a desired quantity. In some embodiments, the temperature of a fermentable composition can be at a temperature in a range from 20° C. to 45° F., from 25° C. to 40° C., or even from 30° C. to 40° C. In some embodiments, primary fermentation can occur for a time period up to 120 hours, e.g., from 1 hour to 120 hours, from 1 hour to 48 hours, from 2 hours to 48 hours, from 10 hours to 30 hours, from 48 hours to 56 hours, from 48 hours to 72 hours, from 45 hours to 75 yours from 45 hours to 88 hours, from 45 hours to 100 hours, or even from 45 hours to 120 hours.

Fermentation can be performed under anaerobic conditions and/or aerobic conditions. For example, fermentation can be performed under aerobic conditions for at least a portion of the fermentation and performed under anaerobic conditions for another portion of fermentation. Alternatively, all of fermentation can be performed under anaerobic conditions or under aerobic conditions. Anaerobic or aerobic conditions are selected based on the microorganism and the “target” biochemical or biochemicals chosen to be produced by a microorganism even though there may de minimis amounts of “non-target” biochemicals that are also produced by the microorganism. Anaerobic conditions means that the fermentation process is conducted without any intentional introduction of oxygen-containing gases such as with equipment like blowers, compressors, etc., that could operate to create an aerobic environment suitable for aerobic fermentation. It is noted that while simply stirring a fermentable composition to keep reactor contents homogenous may or may not introduce a de minimis amount of an oxygen-containing gas such as air in some embodiments, stirring alone may not create conditions that would be considered “aerobic conditions” as used herein. However, if desired, the contents of a fermenter could be mixed using appropriate equipment such that sufficient oxygen is introduced throughout the fermentable composition to create an aerobic environment suitable for aerobic fermentation (see below).

Aerobic conditions means that fermentation is performed with intentional introduction of one or more oxygen-containing gasses (“aeration”) to create an aerobic environment suitable for aerobic fermentation so that oxygen can be consumed by one or more microorganisms and, e.g. in the case of yeast, selectively favor the production of enzymes via an aerobic metabolic pathway as compared to an anaerobic pathway which favors production of biochemicals (e.g., alcohol, organic acids, and the like). A fermentation system may incorporate aeration by including one or more blowers, spargers, gas compressors, mixing devices, and the like, that are in fluid communication with one or more fermentation vessels and that can introduce an oxygen-containing gas (e.g., air) into a fermentable composition during at least a portion of fermentation. For example, an oxygen-containing gas can be sparged into a fermentable composition so that the gas bubbles up and through the fermentable composition and oxygen transfers into the fermentable composition. As another example, an oxygen-containing gas can be introduced into the headspace of a fermenter so that the gas diffuses into the fermentable composition.

Optionally, in addition to aeration, a fermentable composition can be agitated or mixed to facilitate transferring oxygen into and throughout the fermentable composition so as to achieve an aerobic environment. For example, a continuous stirred tank reactor (CSTR) can be used to agitate or mix the fermentable composition. The speed of the stirring mechanism (rpms) can be adjusted based on a variety of factors such as tank size, slurry viscosity, and the like. As mentioned above, in addition to mixing the contents of a composition, mixing can be selected, if desired, to intentionally incorporate oxygen to a fermentable composition to facilitate aerobic fermentation.

A fermentation system can be operated according to batch fermentation, fed-batch fermentation, or continuous fermentation (continuous feed and discharge from a vessel such as a fermenter).

Also, a fermentation system according to the present disclosure can conduct fermentation sequentially or simultaneously with respect to a polysaccharide hydrolysis/saccharification process (e.g., jet-cooking and/or enzymatic hydrolysis). Saccharification and fermentation can occur simultaneously according to what is known as “simultaneous saccharification and fermentation” (“SSF”). Sequential hydrolysis and fermentation can also be referred to as separate hydrolysis and fermentation (SHF).

An example of an SSF process involves forming a slurry that includes a starch-containing grain such as corn. The slurry can be combined with a microorganism to form a fermentable composition so that at least a portion of starch in the fermentable composition is hydrolyzed by one or more enzymes to produce monosaccharides. As the monosaccharides are produced, they can be metabolized by a microorganism into a target biochemical product. For example, sugar (glucose, xylose, mannose, arabinose, etc.) that is generated from saccharification can be fermented into one or more biochemicals (e.g., butanol, ethanol, and the like).

Alternatively, an SHF process may include a dedicated saccharification process that is separate from a fermentation process (either in the same or separate vessel). For example, after forming an aqueous slurry that includes the biomass feedstock (e.g. corn material from a milling system) the aqueous slurry can be subjected to saccharification in one or more slurry tanks to break down (hydrolyze) at least a portion of the polysaccharides, e.g. starch, cellulose, hemicellulose, etc., into oligosaccharides and/or monosaccharides (e.g. glucose, xylose, mannose, arabinose, etc.) that can be used by microorganisms (e.g., yeast) in a subsequent fermentation process.

Saccharification can be performed by a variety of mechanisms. For example, heat and/or one or more enzymes can be used to form one or more monosaccharides by saccharifying one or more oligosaccharides and/or one or more polysaccharides that are present in a polysaccharide such as starch. In some embodiments, a relatively low temperature saccharification process (whether used in SSF or SHF) involves enzymatically hydrolyzing at least a portion of starch in an aqueous slurry at a temperature below starch gelatinization temperatures, so that saccharification occurs directly from the raw native insoluble starch to soluble glucose while bypassing conventional starch gelatinization conditions, which are typically in a range of 57° C. to 93° C. depending on the starch source and polysaccharide type. In some embodiments, saccharification includes using one or more enzymes (e.g., alpha-amylases and/or gluco-amylases) to enzymatically hydrolyze at least a portion of the starch in the aqueous slurry at a temperature of 40° C. or less to produce a slurry that includes glucose. In some embodiments, enzymatic hydrolysis occurs at a temperature in the range of from 25° C. to 35° C. to produce a slurry that includes glucose. Non-limiting examples of converting raw starch to glucose are described in U.S. Pat. No. 7,842,484 (Lewis), U.S. Pat. No. 7,919,289 (Lewis), U.S. Pat. No. 7,919,291 (Lewis et al.), U.S. Pat. No. 8,409,639 (Lewis et al.), U.S. Pat. No. 8,409,640 (Lewis et al.), U.S. Pat. No. 8,497,082 (Lewis), U.S. Pat. No. 8,597,919 (Lewis), U.S. Pat. No. 8,748,141 (Lewis et al.), 2014-0283226 (Lewis et al.), and 2018-0235167 (Lewis et al.), wherein the entirety of each patent document is incorporated herein by reference.

Bioprocessing facility 150 can also include one or more systems downstream from fermentation. For example, after fermentation one or more bioproducts can be separated from beer to form at least one target bioproduct stream (e.g., ethanol) and one or more co-product streams (e.g., whole stillage). A co-product stream can encompass any stillage composition downstream from fermentation after separating one or more bioproducts from beer using separation technologies such as distillation, membrane separation, gas stripping, absorption, and the like. As used herein, a “stillage composition” can include whole stillage, at least one stillage composition derived from whole stillage, and combinations thereof. Non-limiting examples of a stillage composition derived from whole stillage include wet cake, thin stillage, concentrated thin stillage (syrup), defatted syrup, defatted emulsion, clarified thin stillage, distiller's oil, distiller's grain, distiller's yeast, and the like. Non-limiting examples of defatted stillage compositions include one or more defatted streams derived from thin stillage such as defatted syrup, defatted emulsion, and the like.

A separation system can separate a bioproduct from a beer using one or more of distillation, evaporation, separation based on particle size (e.g., filtration), or separation based on density (e.g., centrifugation). In some embodiments, a separation system can include one or more centrifuges (e.g., two-phase vertical disk stack centrifuge, three-phase vertical disk stack centrifuge, filtration centrifuge), one or more decanters (e.g., filtration decanters), one or more filters (e.g., fiber filter, rotary vacuum drum filter, filter device having one or more membrane filters), one or more screens (e.g., a “DSM” screen, which refers to a Dutch State Mines screen or sieve bend screen, and is a curved concave wedge bar type of stationary screen; a pressure screen; paddle screen; rotary drum screen; centrifugal screener; linear motion screen; vacu-deck screen; etc.), one or more brush strainers, one or more vibratory separators, one or more hydrocyclones, one or more presses, combinations of these and the like. Multiple separation systems can be used together and arranged in a parallel and/or series configuration.

Depending on the separation system selected, one or more process input streams can be separated into two or more output streams to produce an output stream that has a higher amount of solids as compared to other output streams. If desired, a separation system can include one or more evaporators and/or one or more dryers to further concentrate an output stream from any of the devices just mentioned.

In some embodiments, at least a portion 158 of one or more bioproducts 157 can be exposed to an anaerobic digestion system 160 to produce one or more effluent compositions. Anaerobic digestion is a process that breaks down organic matter via bacteria in the absence of oxygen to produce biogas, which is a mixture of methane, carbon dioxide, hydrogen sulfide, water vapor, and trace amounts of other gases. Stream 275 includes carbon-dioxide, which can be sequestered and/or further utilized as described herein. Non-limiting examples of organic matter that can be fed to an anaerobic digestion process include one or more stillage compositions. Effluent compositions discharged from an anaerobic digestion process include anaerobic digestion liquid effluent, anaerobic digestion solid effluent, and combinations thereof. Organic nitrogen may be converted to ammonia during anaerobic digestion and be present in an anaerobic digestion digestate composition. If desired the ammonia may be separated from the anaerobic digestion digestate composition via distillation and the like. The separated ammonia can be relatively more concentrated and can be used in making a fertilizer composition according to the present disclosure. A non-limiting example of producing biogas is described in Pub. No. US 2022/0033860 (Bonk et al.), wherein the entirety of each patent document is incorporated herein by reference.

Facility 100, including bioprocessing facility 150, can include one or more systems (not shown) configured to use energy 110 for one or more processes. Such systems are configured to receive at least a portion of the energy 110 from energy generating system 105. For example, energy generating system 105 can include a steam boiler system configured to generate steam that is transported to an evaporator system and/or a distillation system and/or a dryer system via piping within the bioprocessing facility 150. For illustration purposes, in the context of a corn ethanol bioprocessing facility, a distillation system (not shown) can use steam to indirectly heat fermented beer in a heat exchanger and distill ethanol from fermented beer. As yet another example, an evaporator system (not shown) can use steam in a heat exchanger to indirectly heat a back-end stillage stream (e.g., thin stillage or composition derived from thin stillage) to remove water and concentrate the stillage stream. As yet another example, a dryer system (not shown) can be configured to dry at least a portion of one or more bioproducts 157 via steam that is generated by energy generating system 105. The dryer system can be configured to dry a bioproduct using the steam in addition to or instead of using fossil fuel (e.g., natural gas), thereby reducing the carbon intensity of facility 100 even further. For example, a dryer system, such as a rotary steam tube dryer manufactured by the Louisville Dryer Company, can use steam that indirectly contacts and heats a back-end stillage composition to remove moisture from suspended solids (e.g., fiber and/or protein) to make, e.g., distillers' dried grain with solubles (DDGS) and/or a high protein product.

For illustration purposes, additional non-limiting examples of process streams and systems in a corn grain ethanol plant that can be heated (e.g., indirectly or directly via a heated gas or liquid) using thermal energy 112 from energy generating system 105 include front-end streams such as a slurry. For example, a front-end slurry can be exposed to a high-temperature cooking process for starch.

According to the present disclosure, at least a portion of the carbon-dioxide gas in the flue gas 107 is captured. As used herein, “capturing” or “captured” carbon-dioxide gas means that the carbon-dioxide gas generated in facility 100 is not directly released to the atmosphere. Instead, the carbon-dioxide gas is captured for further processing such as by being sequestered in sequestration system 120 and/or further utilized in utilization system 125.

As mentioned above, the composition of flue gas 107, including the concentration of carbon-dioxide gas, can vary depending on a variety of conditions. In some embodiments, concentration of carbon dioxide in a flue gas 107 can be relatively high such that flue gas 107 can be transferred directly to sequestration system 120 and/or utilization system 125. In some embodiments, concentration of carbon dioxide in a flue gas 107 can be relatively low. Optionally, if desired, flue gas 107 can be transferred to a system 106 configured to separate at least a portion of the carbon-dioxide gas from the flue gas 107, thereby concentrating the carbon-dioxide gas into stream 117 before transferring it to sequestration system 120 and/or utilization system 125. Carbon dioxide in a flue gas 107 can be concentrated (purified) utilizing existing technologies such as one or more of chemical solvent systems, physical solvent systems, cryogenic systems, membrane systems, and pressure-swing-absorption (PSA) systems. An example of a chemical solvent system includes an amine separation system. An amine separation system is configured to react amine solvent with carbon-dioxide gas present in flue gas 107 followed by a regeneration process that recovers carbon-dioxide gas separately from amine, thereby providing stream 117 that is a relatively concentrated in carbon-dioxide gas as compared to flue gas 107. Physical solvent systems are configured to absorb carbon-dioxide gas without chemical reaction. Cryogenic systems are configured to condense carbon-dioxide gas out of a vapor stream. Membrane systems are configured to separate carbon-dioxide gas based on molecule size. Pressure-swing-absorption systems are configured to separate carbon-dioxide gas based on physical adsorption in a manner similar to how molecular sieves physically adsorb ethanol.

As mentioned above, energy generating system 105 can produce and deliver energy 115 to the system 106 that is configured to separate at least a portion of the carbon-dioxide gas from the flue gas 107. System 106 can be configured to receive electrical energy 113 and thermal energy 114, depending on the type of energy (steam and/or electricity) and amount of energy needed to operate system 106. Advantageously, the system 106 can reduce or avoid obtaining energy from sources external to facility 100, such as from an external electrical grid, especially energy from sources that use fossil fuel, thereby reducing the relative carbon intensity of facility 100. Because system 106 can receive energy 115 from energy generating system 105, the amount of biomass feedstock 102 used by energy generating system 105 can increase, thereby reducing the carbon intensity of facility 100 even further. As can be seen, as more energy is generated by energy generating system 105 for facility 100, more biomass feedstock 102 is required. In some embodiments, as the electrical energy produced by energy generating system 105 increases, a steam turbine system may utilize a relatively higher input pressure to run the turbine, which can increase amount of biomass feedstock 102 even further.

As mentioned above, carbon-dioxide gas from energy generating system 105 can be sequestered and/or further utilized. In addition to flue gas 107 from energy generating system 105, one or more optional sources of carbon-dioxide gas can be captured and sequestered and/or further utilized as described herein. Non-limiting examples of additional sources of carbon-dioxide gas include stream 155 from a fermentation system in bioprocessing facility 150, stream 159 from anaerobic digestion system 160, and combinations thereof. Referring to FIG. 1, carbon-dioxide gas present in streams 117, 155, and 159 are combined into stream 161. A portion 108 of stream 161 can be sequestered in sequestration system 120 and/or a portion 109 of stream 161 can be further utilized in utilization system 125.

As used herein, “sequestered” and “sequestering” carbon-dioxide gas generated in facility 100 means storing the carbon-dioxide gas instead of releasing it to the atmosphere, thereby enabling a carbon-negative process. The carbon-dioxide gas can be stored in a variety of ways. For example, carbon-dioxide can be transported and stored in underground geological formations. Sequestration system 120 can be configured in a variety of ways depending on how the carbon-dioxide gas in stream 108 is to be sequestered. For example, transportation (e.g., via underground piping, railcar, and/or trucks) of carbon-dioxide to a site remote from facility 100 for storage underground can be more efficient if the carbon dioxide is present as a liquid, which is denser than carbon-dioxide gas. In some embodiments, sequestration system 120 can include one or more compressors to pressurize carbon-dioxide gas present in stream 108 into a liquid phase and/or a supercritical phase. In some embodiments, sequestration system 120 can include equipment configured to dehydrate the stream 108 of carbon-dioxide gas to avoid undue corrosion during transportation and/or storage. Carbon-dioxide may also be reacted to form solid carbon and carbon compounds that may be sequestered.

Utilization system 125 can use carbon-dioxide gas in a variety of ways. In some embodiments, utilizing carbon-dioxide gas in a utilization system means using the carbon-dioxide gas in a carbon-neutral manner A non-limiting example of “utilizing” carbon-dioxide gas in stream 109 in a carbon-neutral manner includes reacting at least one reactant and at least a portion of the carbon-dioxide to form at least one reaction product. A wide variety of reaction products can be produced. Non-limiting examples of a reaction product include at least one of carbon monoxide, methane, methanol, formate, formic acid, ethanol, ethylene, propylene, and combinations thereof. In some embodiments, the carbon-dioxide gas from stream 109 can be used for the production of green chemicals. A “green” chemical is produced using at least one renewable feedstock and/or at least one source of renewable energy. Non-limiting examples of renewable energy sources include wind power, solar power, hydroelectric power, combustion of biomass, and combinations thereof. An example of producing a green chemical using carbon-dioxide gas from stream 109 is methane (also referred to as “eMethane”), which can be produced using green hydrogen. It is noted that the carbon-dioxide gas from stream 109 can be prepared as desired prior to being utilized. For example, it can be one or more of heated, cooled, pressurized (e.g., into a liquid), purified, and the like.

Utilization system 125 can include at least a chemical production system (not shown) that is co-located with the bioprocessing facility 150. The chemical production system can be in fluid communication with energy generating system 105 to receive carbon-dioxide gas via stream 109. A non-limiting example of using carbon-dioxide gas from stream 109 in utilization system 125 for the production of chemicals is illustrated in FIG. 3 (discussed below). A non-limiting example of utilizing carbon-dioxide gas is described in U.S. Ser. No. 18/124,972 (Carlson et al.) filed on Mar. 22, 2023, titled “Systems and methods FOR producing ONE OR MORE CHEMICALS USING CARBON DIOXIDE PRODUCED BY fermentation,” wherein the entirety of each patent document is incorporated herein by reference.

In some embodiments, utilization system 125 can generate energy 130, which includes electrical energy 131 and thermal energy 132. Optionally, facility 100 is configured to use at least a portion of energy 130 (electrical energy 131 and/or thermal energy 132) generated by utilization system 125. An example of producing energy 130 for bioprocessing facility 150 is described below in connection with FIG. 3.

FIGS. 2A-2C illustrate how a facility 200 can reduce its carbon intensity at a bioprocessing facility 250 such as a corn ethanol bioprocessing facility, by capturing atmospheric carbon-dioxide gas in a biomass feedstock and generating energy via combustion of the biomass feedstock. To help illustrate this, carbon dioxide production and absorption are mathematically “netted” to determine the carbon intensity (CI) of a process. In FIGS. 2A-2C, this is shown numerically with units of CI (g CO2/MJ or g/MJ), which is shorthand for grams of CO2 per mega joule of energy. This is shown as g CO2/MJ where the energy is normalized to the energy in ethanol (bioethanol) produced in bioprocessing facility 250. As illustrated herein, industrial corn grown for ethanol production and animal feed has the potential to be an extremely effective decarbonization product and an option or alternative to conventional direct-air capture technologies. To help illustrate the “carbon sink” or decarbonization impact of facility 200, a solid-line box 299 is drawn to highlight the carbon-dioxide flow into and out of the box 299. In the non-limiting example shown in FIG. 2C and discussed below, approximately 320 g CO2/MJ flows into the box 299 while only a total of 150 g CO2/MJ (115 g CO2/MJ+35 g CO2/MJ) flows out, for a reduction of 170 g CO2/MJ from the atmosphere.

A base scenario will be described with reference to FIG. 2A. A non-limiting embodiment of decarbonization according to the present disclosure is illustrated in FIGS. 2B and 2C. FIG. 2B is a process-flow diagram for comparison to the base scenario in FIG. 2A. FIG. 2C is a more detailed schematic of the process-flow diagram in FIG. 2B showing additional features.

In corn farming 201, an average acre of corn absorbs approximately 13.4 MT of CO2 during each annual growing season. When the corn grain produced from the acre of corn is used for ethanol production in bioprocessing facility 249, this equates to approximately 320 g CO2 absorbed 206 by the entire corn plant via corn photosynthesis for each MJ of energy content of the ethanol produced (320 g CO2/MJ) from the corn grain. It is noted that 320 g CO2 absorbed 206 by the entire corn plant is an estimate and may be different (e.g., higher) depending on data used for calculations. Approximately half of the carbon absorbed by a corn plant is contained in the corn grain, about 150 g CO2/MJ from the acre of corn plants, while about 170 g CO2/MJ is contained in the remainder of the corn plant, or corn stover. As shown in FIG. 2A, corn farming 201 on average emits 202 approximately 30 g CO2/MJ in anthropogenic farming emissions, with most of the emissions coming from nitrogen fertilizer production and application, assuming that all of the fertilizer is produced and applied utilizing fossil resources. As shown in FIG. 2A, corn farming 201 on average emits 207 approximately 170 g CO2/MJ in biogenic emissions as corn stover decomposition. These emissions can be reduced by implementing climate smart agriculture practices such as no-till, cover crops, 4R fertilizer application, and utilization of blue and green nitrogen fertilizers.

As shown in FIGS. 2B and 2C, such practices in corn farming 221 produces corn plants 205 and can conservatively reduce baseline farming emissions in corn farming 201 as shown in FIG. 2A by a reduction 203 of 15 g CO2/MJ in corn farming 221 utilizing climate-smart agriculture practices as shown in FIGS. 2B and 2C that emit 209 15 g CO2/MJ in farming emissions. Also, as shown in FIGS. 2B and 2C, no-till farming can additionally conservatively sequester 204 another 15 g CO2/MJ carbon in soil biomass in the form of soil organic carbon (SOC), thereby reducing the corn stover decomposition by 15 g CO2/MJ so that corn farming 221 as shown in FIG. 2B emits 211 about 115 g CO2/MJ in corn stover decomposition. As discussed below, using corn stover as biomass feedstock 212 for solid fuel for energy generating system 220 can further reduce the corn stover decomposition by capturing 40 g CO2/MJ in flue gas 228. As shown in FIGS. 2B and 2C, the reductions of corn stover decomposition by 15 g CO2/MJ and 40 g CO2/MJ can reduce corn stover decomposition from 170 g CO2/MJ to 115 g CO2/MJ in corn stover decomposition.

Cover crops are typically off-season crops planted to cover the soil to protect and enhance the soil when the primary crop (e.g., corn) is not being grown. Cover crops manage soil erosion, soil fertility, soil quality, water, weeds, pests, diseases, and biodiversity in the agroecosystem and, among other potential CI benefits, help build soil organic carbon and reduce fertilizer utilization. Cover crops also increase the CO2 capture via CO2 absorption by the cover crops. The cover crops also produce additional biomass that may be used for fuel as biomass feedstock for combustion. These additional CI benefits are not accounted for in the system illustrated in FIGS. 2B and 2C.

No-till farming is an agricultural technique for growing crops without disturbing the soil through tillage. Benefits of no-till farming include increased water penetration, retention of organic matter and retention of nutrients. Among other potential CI benefits, no-till farming can sequester 204 carbon in the soil as soil organic carbon and reduces fertilizer utilization.

4R fertilization refers to utilizing the right fertilizer source, at the right rate, at the right time, and in the right place. It is a component of precision agriculture with potential CI benefits such as reducing fertilizer utilization and shifting to greener (lower CI) sources of fertilizer. Blue and green nitrogen fertilizers are nitrogen fertilizers with lower CI than traditional nitrogen fertilizers. Traditional nitrogen fertilizers are produced from natural gas by stripping hydrogen from the methane molecule and combining the hydrogen with atmospheric nitrogen. The process evolves a large amount of CO2. Blue nitrogen fertilizers are produced in a process that captures the evolved CO2 and sequesters it or uses it for another green purpose. Green nitrogen fertilizers are produced in a process that sources hydrogen from a non-fossil process such as from electrolysis utilizing green electricity.

As shown in FIG. 2A, bioprocessing facility 249 can emit a total of approximately 60 g CO2/MJ of which 35 g CO2/MJ is biogenic from the carbon dioxide produced during fermentation and 25 g CO2/MJ is anthropogenic from the use of fossil energy. The fossil energy emissions are mostly from natural gas emissions 266 (21 g CO2/MJ) for steam and co-product drying, as well as grid electricity emissions 267 (4 g CO2/MJ) for motors and pumps. These fossil emissions can be reduced or eliminated by utilizing biomass feedstock 212 in an energy generating system 220 such as a combined heat and power (CHP) system that includes a solid fuel boiler to combust biogenic fuels such as corn stover or other waste biomass to produce energy 225 such as thermal energy 227 (e.g., biogenic steam). This biogenic steam can be used for process heat. In addition, a steam turbine generator can be deployed in a combined heat and power configuration to utilize some of the biogenic steam to produce electrical energy 226. Utilizing corn stover or other waste biomass for thermal energy 227 and electrical energy 226 is a mostly carbon neutral process as the carbon stored in the corn stover or waste biomass is biogenic and can be absorbed by corn in the growing season. As shown in FIGS. 2B and 2C, the equivalent of 130 g CO2/MJ of the available stover is left behind and sequestered to contribute to SOC (15 g CO2/MJ) and emitted 211 to the atmosphere (115 g CO2/MJ) via decomposition, while the equivalent of 40 g CO2/MJ of the stover is burned in the combined heat and power facility and captured via flue gas 228. If more of the stover is collected for heat and power, a larger CI reduction will be realized. There is a balance between leaving stover for soil health and power production. In terms of calculating the net reduction of CO2 by combusting these feedstocks, as shown in FIG. 2C, combusting these feedstocks for energy not only reduces existing natural gas emissions 266 and grid electricity emissions 267 equivalent to a reduction of approximately 25 g CO2/MJ, but also creates a flue gas 228 that is a concentrated biogenic CO2 exhaust stream of 40 g CO2/MJ, which may be captured, for a total of 65 g CO2/MJ net reduction.

In some embodiments, as shown in FIG. 2C, the 40 g CO2/MJ in the flue gas 228 that is captured from combustion can be purified in system 230 utilizing technologies such as amine separation systems. System 230 is configured to separate at least a portion of the carbon-dioxide gas from the flue gas 228 to form stream 238, which is relatively more concentrated in carbon-dioxide gas as compared to flue gas 228. In some embodiments, as shown in FIG. 2C, energy generating system 220 can produce and deliver energy 215 to the system 230. System 230 can be configured to receive electrical energy 213 and thermal energy 214, depending on the type of energy (steam and/or electricity) and amount of energy needed to operate system 230. Carbon-dioxide gas in stream 228 from energy generating system 220 can be sequestered and/or further utilized. Referring to FIG. 2C, carbon-dioxide gas resulting from combustion of ethanol stream 255 is combined with carbon-dioxide streams 265 and 271 to form stream 275 and combined with stream 238 to form stream 261. A portion 281 of stream 261 can be sequestered via sequestration system 280 as described above relative to sequestration system 120 of FIG. 1, and/or a portion 282 of stream 261 can be further utilized in utilization system 285 as described above relative to utilization system 125 of FIG. 1.

It is noted that if more corn stover is used biomass feedstock 212 instead of being left to decompose, then corn farming 221 would emit 211 less in corn stover decomposition and reduce carbon-dioxide emissions even more by capturing more carbon-dioxide in flue gas 228. For example, if additional corn stover is combusted, instead of being decomposed, to provide additional thermal energy (e.g., for one or more process internal and/or external to facility 200) and/or to generate green electricity (e.g., for use internal and/or external to facility 200) via a steam turbine the total amount of CO2 that could be captured from the combined heat and power step could be increased from 40 g CO2/MJ while the CO2 emitted 211 due to decomposition could be reduced from 115 g CO2/MJ. The sum of 115 g CO2/MJ from decomposition and 40 g CO2/MJ from flue gas 228 is 155 g CO2/MJ so the CO2 that could be captured from the combined heat and power step could be theoretically increased from 40 g CO2/MJ up to 155 g CO2/MJ. An example of combusting more corn stover in energy generating system 220 for a system internal to facility 200 is an amine separation system to purify flue gas 228 as discussed above with respect to system 106 in FIG. 1.

In some embodiments, utilization system 285 can generate energy 233, which includes electrical energy 231 and thermal energy 232. Optionally, facility 200 is configured to use at least a portion of energy 233 (electrical energy 231 and/or thermal energy 232) generated by utilization system 285.

As mentioned above with respect to FIG. 2A, another source of biogenic emissions may be created during the ethanol production process in the bioprocessing facility 249 as the starch and fiber from the corn kernel is fermented by yeast into ethanol in stream 255. The yeast evolves biogenic CO2 as part of its metabolism. This fermentation CO2 stream tends to be very pure. In FIGS. 2B and 2C, biomass feedstock 251 includes corn grain that is derived from corn plants 205, and instead of exhausting carbon-dioxide gas to atmosphere via stream 254, the biogenic CO2 from bioprocessing facility 250 that is generated from the production of ethanol in stream 255 is easily captured in stream 265 using existing technologies and sequestered in sequestration system 280. As shown in FIGS. 2B and 2C, capturing and sequestering the fermentation CO2 could result in approximately 35 g CO2/MJ of biogenic CO2 being permanently sequestered that would have otherwise been released into the atmosphere via stream 254. As shown in FIG. 2C, in addition to or instead of sequestration, biogenic CO2 could be further utilized in utilization system 285.

The final ethanol in stream 255 contains approximately 70 g CO2/MJ of the 150 g CO2/MJ biogenic carbon in the corn grain. (70 g CO2/MJ in the ethanol=150 g CO2/MJ in the corn−35 g CO2/MJ biogenic CO2 evolved in fermentation−45 g CO2/MJ in co-products). As shown in FIGS. 2A and 2B, when the ethanol in stream 255 is combusted, the resulting biogenic CO2 can be captured 257 instead of exhausting 256 to atmosphere. Capturing this CO2 is most easily done in stationary industrial processes such as electricity generation and heavy-duty transportation (e.g., ships, trains, heavy trucks) which can support the necessary capture equipment. Concentrated CO2 in the exhaust stream or an ethanol burning process can be purified utilizing existing technologies such as an amine separation system. The captured CO2 may be sequestered in sequestration system 280. Capturing and sequestering all of the ethanol combustion CO2 could result in approximate 70 g CO2/MJ of biogenic CO2 being permanently sequestered that would have otherwise been released to the atmosphere. In addition to sequestration, biogenic CO2 could be utilized in utilization system 285.

In addition to ethanol, the bioprocessing facility 250 produces other bioproducts 260, e.g., co-products, that together account for 45 g CO2/MJ, which may be released 263 to the atmosphere. The main co-product of the ethanol production process is called distiller's grain (DG) which can be used as an animal feed. Most of the carbon in this product will be released as CO2 during animal digestion and would be difficult to capture with existing technologies. However, according to the present disclosure, some or all 264 of the DG could be used in an anaerobic digestion system 270 to capture carbon, while the remaining DG could be used as animal feed, which would be digested as described in FIG. 2A and produce carbon dioxide in stream 263. Also, there are streams in the bioprocessing facility 250 upstream of DG production that are well suited for anaerobic digestion. For example, a portion of a stillage stream, e.g., thin stillage or a stream derived from thin stillage, may be diverted from the DG production and converted to biomethane and CO2 through using existing anaerobic digestion technologies. The biomethane can then be used to replace existing fossil fuel applications such as transportation, home heating, electrical generation, etc. The CO2 emissions can be captured and sequestered in sequestration system 280. In one example, referring to FIG. 2B, all of the remaining bioproducts can be anaerobically digested to produce biogas. If this biogas is combusted such that all of the post combustion CO2 is sequestered the total amount of carbon intensity reduction is 45 g CO2/MJ. However, because the bioproducts in this example are not used as animal feed the 10 g CO2/MJ coproduct credit shown in FIG. 2A does not apply. Referring to FIG. 2C, if only a portion of the bioproducts are anaerobically digested to produce biogas, the portion of the bioproducts that are used as animal feed still contribute 35 g CO2/MJ of emissions. If the biogas is combusted such that all of the post combustion CO2 is sequestered the total amount of carbon intensity reduction is 10 g CO2/MJ, or if only the carbon dioxide in the biogas is sequestered the carbon intensity reduction is only 2 g CO2/MJ. In addition to sequestration, biogenic CO2 could be utilized in utilization system 285.

FIG. 3 shows a non-limiting embodiment of a facility 300 that includes a bioprocessing facility 310 shown as a corn ethanol bioprocessing facility. As shown in FIG. 3, bioprocessing facility 310 receives corn grain feedstock 312. Bioprocessing facility 310 includes one or more fermentation vessels that produce at least one target biochemical 314 shown as ethanol along with a stream 316 concentrated in carbon dioxide gas. Facility 300 is configured to use at least a portion of energy 336 from energy generating system 330, shown as a combined heat and power (CHP) system, in the one or more systems in the bioprocessing facility 310. Non-limiting examples of such systems include an evaporator system, a distillation system, a dryer system, and combinations thereof. Energy generating system 330 burns fuel to generate energy 336 and flue gas 334. A portion of flue gas 334 can be sequestered as described above relative to sequestration system 120 of FIG. 1, and/or a portion of flue gas 334 can be further utilized as described above relative to utilization system 125 of FIG. 1. An example of utilizing flue gas 334 is described below with respect to utilization system 355.

Facility 300 integrates a utilization system 355 with bioprocessing facility 310. Utilization system 355 is also configured to use “green” carbon dioxide and “green” hydrogen in an exothermic reaction to make water vapor stream 456 and methane stream 458 using carbon dioxide 316 from bioprocessing facility 310. At least a portion of the thermal energy 361 from the exothermic reaction is used in the bioprocessing facility 310. Energy from the exothermic reaction offsets energy needed from energy generating system 330 such that the mass flow rate of flue gas 334 from energy generating system 330 is reduced due to less energy needing to be generated by energy generating system 330. The energy generating system 330 can have a corresponding reduction in biomass feedstock 332, e.g., corn stover. As shown, the carbon dioxide in flue gas 334 is recovered and used in utilization system 355. The carbon dioxide that is present in flue gas 334 can be cleaned to produce a relatively more pure feedstock stream 345 for the utilization system 355. This cleaning may be performed with one or more additional unit operations 340 such as e.g., amine scrubbing, pressure swing adsorption, cryogenic distillation, or membrane separation. The additional carbon-dioxide from stream 345 increases the output of utilization system 355 both in terms of product, e.g., methane, and excess energy which further reduces the energy needed from system 330. It is noted that an equilibrium can be reached among bioprocessing facility 310, energy generating system 330, and utilization system 355, which can depend on one or more factors such as the excess energy generated by utilization system 355.

Facility 300 uses not only “green” carbon dioxide from energy generating system 330 and bioprocessing facility 310, but also “green” hydrogen 357 from optional electrolysis system 370. As shown in FIG. 3, electrolysis system 370 is configured to form hydrogen 357 and oxygen 376 from water 374. Facility 300 integrates at least a portion 378 of oxygen 376 produced in electrolysis system 370 with the combustion processes associated in energy generating system 330. If desired, any excess oxygen 380 that may be present can be vented. Providing the portion 378 of oxygen to energy generating system for combustion can produce flue gas 334 that is relatively much more concentrated in carbon dioxide gas as compared to using atmospheric air for combustion. This results in a flue gas 334 that is essentially pure carbon-dioxide gas on a dry basis and is much easier to capture and use as a feedstock for sequestering and/or utilizing (e.g., in utilization system 355) as described previously. For example, this can provide even more carbon dioxide to feed the utilization system 355, which increases the output of utilization system 355 both in terms of product, e.g., methane, and excess energy which further reduces the energy needed from system 330. This in turn reduces the amount of biomass feedstock 332 needed by the energy generating system 330 of the bioprocessing facility 310 and the amount of flue gas 334. This may also reduce the processing, if any, to clean up of the flue gas 334 before the carbon dioxide in stream 345 is used in the methanation process. As mentioned above, an equilibrium can be reached among bioprocessing facility 310, energy generating system 330, and utilization system 355 in terms of energy generation and use.

Electricity 372 is used by electrolysis system 370 for the electrolysis of water. In some embodiments, when “green” hydrogen is to be produced, at least a portion of the electricity 372 is generated via one or more sources of renewable energy chosen from wind power, solar power, hydroelectric power, combustion of biomass, and combinations thereof. In some embodiments, hydrogen can also be produced by a carbon-free technology such as nuclear power.

1. A method of capturing atmospheric carbon-dioxide gas, wherein the method comprises:
generating energy via combustion of a biomass feedstock, wherein the combustion of the biomass feedstock produces a flue gas comprising carbon-dioxide gas;
providing at least a portion of the energy to a bioprocessing facility (directly not via a grid); and
capturing at least a portion of the carbon-dioxide gas in the flue gas.

2. The method of claim 1, wherein capturing at least a portion of the carbon-dioxide gas in the flue gas comprises concentrating at least a portion of the carbon-dioxide gas.

3. The method of claim 2, wherein the concentrating at least a portion of the carbon-dioxide gas comprises separating at least a portion of the carbon-dioxide gas from the flue gas via one or more systems that receive at least a portion of the energy generated via combustion of the biomass feedstock.

4. The method of claim 1, wherein providing at least a portion of the energy to a bioprocessing facility comprises providing steam to a dryer system configured to dry a bioproduct using the steam.

5. The method of claim 1, further comprising:
reacting at least one reactant and at least a portion of the carbon-dioxide gas captured from the flue gas to form the at least one reaction product via an exothermic reaction; and
using at least a portion of thermal energy from the exothermic reaction in the bioprocessing facility.

6. The method of claim 5, wherein the at least one reactant comprises hydrogen, wherein at least a portion of the hydrogen is produced via electrolysis of water, wherein the electrolysis of water produces hydrogen and oxygen, and further comprising providing oxygen from the electrolysis of water for at least a portion of oxygen used in the combustion of a biomass feedstock.

7. The method of claim 5, wherein the at least one reaction product is chosen from methane, methanol, formate, formic acid, ethanol, ethylene, propylene, carbon monoxide, and combinations thereof.

8. The method of claim 6, wherein electricity is used for the electrolysis of water, and wherein at least a portion of the electricity is generated via one or more sources of renewable energy chosen from wind power, solar power, hydroelectric power, combustion of biomass, and combinations thereof.

9. The method of claim 1, wherein the at least a portion of the carbon-dioxide gas is sequestered.

10. The method of claim 1, wherein the at least a portion of the carbon-dioxide gas is utilized.

11. The method of claim 1, wherein the energy is chosen from electrical energy, thermal energy, and combinations thereof.

12. The method of claim 1, wherein the bioprocessing facility comprises a corn-ethanol bioprocessing facility.

13. The method of claim 1, wherein the biomass feedstock comprises agricultural residue.

14. The method of claim 1, wherein the biomass feedstock comprises corn stover.

15. The method of claim 1, further comprising capturing at least a portion of carbon-dioxide gas produced by one or more processes in the bioprocessing facility.

16. The method of claim 15, wherein the or more processes are chosen from fermentation of at least one biomass feedstock, anaerobic digestion of at least one stillage composition, and combinations thereof.

17. A facility adapted to capture atmospheric carbon-dioxide gas, wherein the facility comprises:
at least one energy generating system configured to generate energy via combustion of a biomass feedstock and produce a flue gas comprising carbon dioxide, wherein the facility is adapted to capture at least a portion of the carbon-dioxide gas in the flue gas; and
a bioprocessing facility co-located with the at least one energy generating system, wherein the bioprocessing facility comprises:
a fermentation system configured to ferment a fermentable composition and generate at least one target biochemical and carbon-dioxide gas; and
one or more systems configured to use energy for one or more processes in the bioprocessing facility, wherein at least one of the one or more systems are configured to receive at least a portion of the energy from the at least one energy generating system (directly not via a grid).

18. The facility of claim 17, further comprising a system configured to separate at least a portion of the carbon-dioxide gas from the flue gas.

19. The facility of claim 18, wherein the system is configured to receive at least a portion of the energy generated by the at least one energy generating system.

20. (canceled)

21. The facility of claim 17, further comprising:
a chemical production system that is co-located with the bioprocessing facility, wherein the chemical production system is in fluid communication with the at least one energy generating system to receive at least a portion of the carbon-dioxide gas from the flue gas, wherein the chemical production system comprises:
a source of at least one reactant that can react with the carbon-dioxide gas via an exothermic reaction to produce at least one reaction product; and
a reaction vessel configured to react the at least one reactant and carbon dioxide to form the at least one reaction product via the exothermic reaction,
wherein the chemical production system is configured to transport thermal energy from the exothermic reaction, wherein the facility is configured to use at least a portion of the thermal energy to generate electricity for the bioprocessing facility and/or wherein the facility is configured to use at least a portion of the thermal energy in the one or more systems in the bioprocessing facility, wherein the one or more systems are chosen from an evaporator system, a distillation system, a dryer system, and combinations thereof.

22. (canceled)

23. (canceled)

24. (canceled)

25. (canceled)
